(19) United States
(12) Patent Application Publication
(10) Pub. No.: US 2009/0112085 A1
(43) Pub. Date: Apr. 30, 2009

(54) PREOPERATIVE LOCALIZATION WIRE

(75) Inventor: Peter R. Eby, Seattle, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: UNIVERSITY OF WASHINGTON, Seattle, WA (US)

(21) Appl. No.: 12/255,482

(22) Filed: Oct. 21, 2008

Related U.S. Application Data
(60) Provisional application No. 60/983,733, filed on Oct. 30, 2007.

(51) Int. Cl. A61B 5/05

(52) U.S. Cl. 600/424

(57) ABSTRACT

The present invention discloses a novel preoperative localization wire. The localization wire comprises a J-shaped end; a branch point proximal to the J-shaped end, wherein a barb is attached to the wire at the branch point; and a locking device distal to the J-shaped end. The localization wire is resilient, allowing passage of the wire through a localization needle.

### CROSS-REFERENCE TO RELATED APPLICATION

This application claims the benefit of Provisional Application No. 60/983,733, filed Oct. 30, 2007, which is expressly incorporated herein by reference in its entirety.

### FIELD OF INVENTION

The present invention relates to preoperative localization wires and methods for deploying preoperative localization wires.

### BACKGROUND

Except for skin cancers, breast cancer is the most frequently diagnosed cancer among women. Approximately 240,000 cases of breast cancer were diagnosed in the United States in 2007. Many breast cancers and other breast lesions are discovered as a palpable abnormality by patients or their providers during a breast exam. Diagnostic imaging and biopsy of these lesions may reveal high-risk or malignant findings that require surgical excision. Typically, palpable lesions can be removed in the operating room by a surgeon using only touch for guidance.

However, many breast lesions are discovered during screening mammography examinations before the lesion can be reliably identified by touch. Many of these lesions are small and amenable to breast conserving surgery. Typically, when small lesions are to be removed a radiologist will place a wire or wires percutaneously at the site of the breast lesion using sonographic, mammographic, or MRI guidance. The surgeon then uses the wires as a guide for locating the lesion during surgery to allow for complete resection of the lesion while preserving normal breast tissue. The localization wires are therefore critical for identifying the extent of the abnormal tissue, and important for successful removal of the lesion.

The use of localization wires is increasing with the increasingly early detection of breast cancers and the popularity of breast-conserving treatments. Localization wires may also be used as guides for the surgical removal of non-cancerous lesions.

Two types of localization wires are currently in common use: the “Homer” or J-shaped localization wire 100 (FIG. 1) and the “Kopans” or barbed localization wire 200 (FIG. 2). These wires are thin and flexible, and are typically made from stainless steel or alloys with shape memory. To position the localization wire, the wire is typically slidably inserted into a hollow deployment needle that provides stiffness and support during placement. After a localization wire is positioned for optimal resection, the deployment needle is removed and the wire is left in the tissue.

FIG. 1A shows a conventional J-shaped localization wire 100, which is flexible and has shape memory. The wire 100 has a locking device 110 that is used to advance the wire 100 and to indicate when the J-shaped hook end 105 has been completely deployed. The locking device 110 mates with the clear plastic hub 130 disposed on the end of the deployment needle 140. FIG. 1B shows a close-up view of the J-shaped end 105 of the wire 100 extending from the deployment needle 140.

The J-shaped localization wire 100 has the advantage that it is retrievable or removable after it has been deployed, which allows repositioning of the wire 100 if the initial position turns out to be suboptimal. However, the J-shaped wire 100 may be inadvertently and prematurely extracted during an operation, for example if the surgeon pulls on the wire 100 with too much force. This can result in a failed surgery, and expensive repeat visits to the operating room.

FIG. 2A shows a conventional barbed localization wire 200. The barbed localization wire 200 is similar to the J-shaped wire 100 described above, but it includes a barbed tip 205, and may generally include a locking device 210 that engages the deployment needle hub 130. In comparison to the J-shaped wire 100, the barbed localization wire 200 is much less likely to be inadvertently extracted because the barbed tip 210 resists extraction of the wire 200.

However, the relatively sharp tip 205 of the barbed localization wire 200 allows the wire to advance deeper into the breast after the wire 200 has been positioned. There are reports of the entire wire disappearing inside the breast and migrating to distant and sometimes critical parts of the body. In addition, because the localization wire is so thin and the barb tip 205 is located at the very distal end of the wire 200, these wires 200 have also been known to fracture at the apex of the barbed tip 205 during surgery. Wire fracture and migration may require at least two additional and expensive operations: one to find the wire or wire fragment and remove it, and perhaps a second to remove the lesion after a new wire has been placed.

Currently, there are varieties of J-shaped and barbed localizations wires on the market for breast lesion localization applications. These wires have also been used for other surgical applications, such as localization of a small nodule in the lung. With the increasing interest in screening for lung cancer, it is expected that many small, indeterminate lesions may be discovered that could require surgical removal.

Therefore, there is a need for a preoperative localization wire that would be retrievable or removable after the wire is deployed if the position turns out to be suboptimal and that would resist migration or significant movement after the localization wire is placed in a desired location. The present invention seeks to fulfill this need and provide further related advantages.

### SUMMARY OF THE INVENTION

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present invention provides a preoperative localization wire, comprising:

a J-shaped end;

a branch point proximal to the J-shaped end, wherein a barb is attached to the wire at the branch point; and

a locking device distal to the J-shaped end,

wherein the wire is resilient, allowing passage of the wire through a needle.

The J-shaped end may have a radius from about 3 mm to about 6 mm. In one embodiment, the J-shaped end has a radius of about 5 mm.

The branch point can be from about 2 mm to about 8 mm proximal to the J-shaped end bend. In one embodiment, the branch point is from about 2 mm to about 3 mm proximal to the J-shaped end bend.

The preoperative localization wire of the present invention may be made of a suitable material with shape memory property. In one embodiment, the wire is made of stainless steel. In another embodiment, the wire is made of shape memory alloy.

In another aspect, the present invention provides a method for deploying a preoperative localization wire, including the steps of:

(a) providing a preoperative localization wire comprising:

a J-shaped end;

a branch point proximal to the J-shaped end, wherein a barb is attached to the wire at the branch point; and

a locking device distal to the J-shaped end;

(b) providing a localization needle having a hub at one end and a tip at the opposite end;

(c) loading the preoperative localization wire into the localization needle, wherein the localization wire is resilient, allowing passage of the localization wire through the localization needle, and wherein the J-shaped end and the barb of the localization wire are compressed;

(d) placing the localization needle into a tissue with the tip of the localization needle at a desired location;

(e) advancing the preoperative localization wire with the locking device until the locking device contacts the hub, wherein the hub accepts the locking device, and wherein the J-shaped end is deployed and the barb remains compressed;

(f) removing the locking device; and

(g) deploying the barb by pulling back the localization needle while fixing the localization wire.

In one embodiment, the method of the present invention may further include the step of confirming the position of the localization wire to be at the desired location prior to removing the locking device. In another embodiment, the method further comprises retracting and repositioning the localization wire to place the wire at the desired location after confirming the position of the localization wire.

The method of the present invention may further include the step of removing the localization needle from inside the tissue.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows a conventional J-shaped localization wire;

FIG. 1B shows an enlarged view of the J-shaped end of the J-shaped localization wire shown in FIG. 1A;

FIG. 2A shows a conventional barbed localization wire;

FIG. 2B shows an enlarged view of the barbed end of the barbed localization wire shown in FIG. 2A;

FIG. 3A shows a localization needle;

FIG. 3B shows a representative localization wire in accordance with the teachings of the present invention;

FIG. 3C shows a localization needle loaded with a localization wire of the present invention with the compressed J-shaped end and barb;

FIG. 3D shows a localization needle loaded with a localization wire of the present invention with the deployed J-shaped end, the compressed barb, and the locking device contacting the hub of the needle;

FIG. 3E shows a localization needle loaded with a localization wire of the present invention with the deployed J-shaped end, the compressed barb, and the locking device removed;

FIG. 3F shows a localization needle partially retracted on a localization wire of the present invention with the deployed J-shaped end and barb, and the locking device removed; and

FIG. 3G shows a localization wire of the present invention in full deployment in a tissue with the deployed J-shaped end and barb and the localization needle completely removed.

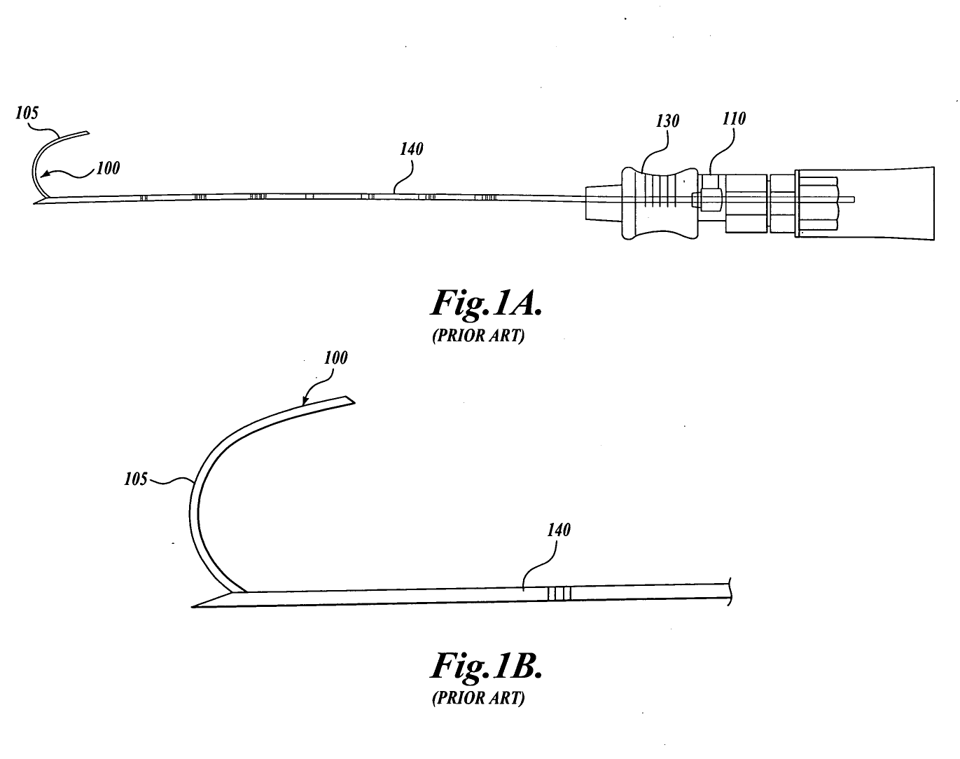

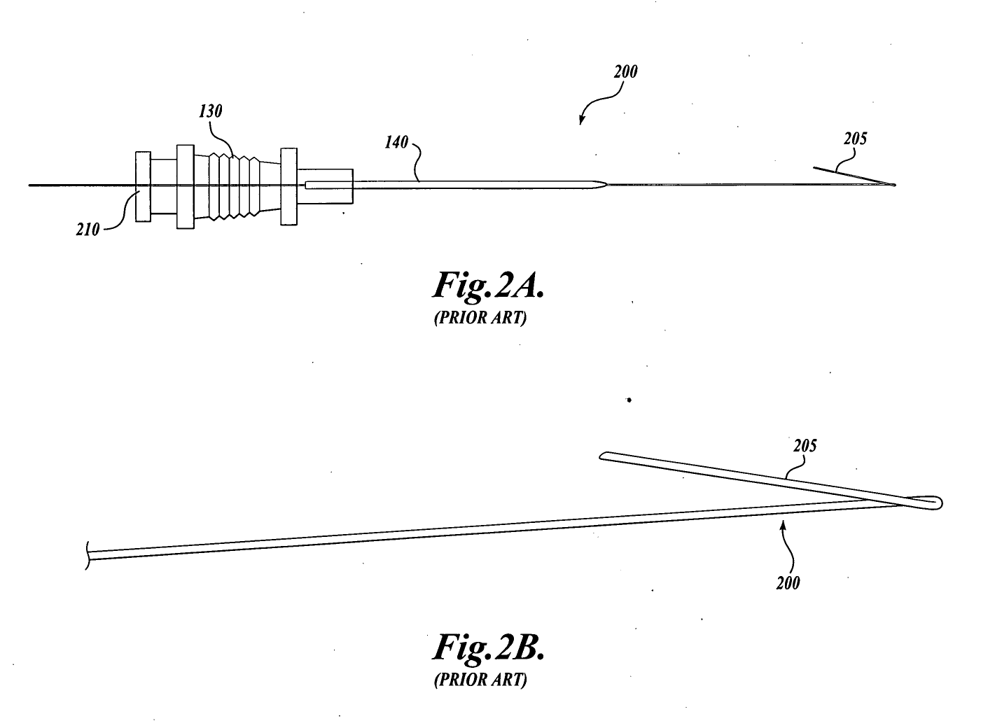

### DETAILED DESCRIPTION OF THE INVENTION

In one aspect, the present invention provides preoperative localization wires.

In one embodiment, the preoperative localization wire comprises:

a J-shaped end;

a branch point proximal to the J-shaped end, wherein a barb is attached to the wire at the branch point; and

a locking device distal to the J-shaped end,

wherein the wire is resilient, allowing passage of the wire through a localization needle.

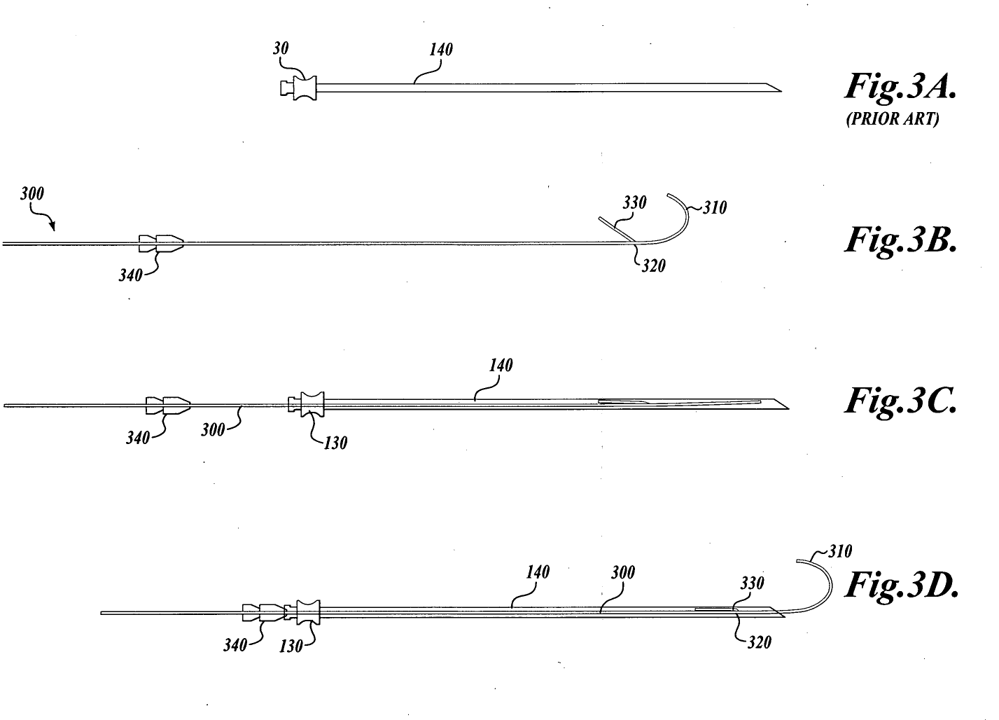

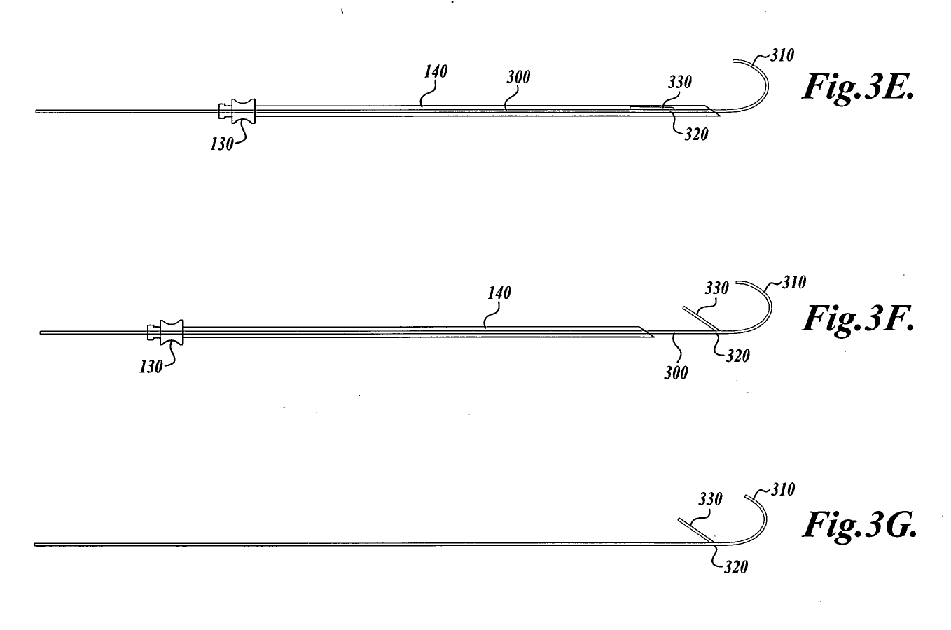

A conventional deployment needle 140 is shown in FIG. 3A. A representative preoperative localization wire 300 in accordance with the teachings of the present invention is illustrated in FIG. 3B. The localization wire 300 has a J-shaped end 310, similar to the J-shaped end 105 of the Homer wire 100 discussed above. A barb 330 is attached to the wire 300 at a branch point 320 disposed proximally from the J-shaped end 310. The barb 330 extends proximally, at an acute angle, thereby tending to retain the wire 300 in a deployed position after it has been inserted into the patient.

The radius of the J-shaped end 310 of the localization wire 300 is preferably between about 3 mm to about 6 mm, and more preferably the radius of the J-shaped end 310 is about 5 mm.

The branch point 320 for the barb 330 is preferably located approximately about 2 mm to about 8 mm proximal to the J-shaped end 310 bend, or about 10 mm to 16 mm from the tip end of the J-shaped end 310. In a currently preferred embodiment, the branch point 320 is about 5 mm proximal of the J-shaped end bend 310, or about 15 mm from the tip of the J-shaped end 310.

FIG. 3C shows a combination including the deployment needle 140 loaded with the localization wire 300. The J-shaped end 310 and the barb 330 are compressed. The localization wire 300 includes a threaded locking device 340 attached to the wire. The locking device 340 retains the J-shaped end 310 of the wire 300 in the deployment needle 140 as the wire 300 is advanced to the desired position.

The locking device 340 allows the J-shaped end 310 of the wire 300 to deploy from the needle 140 when it contacts the hub 130 and prevents deployment of the barb 330 until the correct position has been confirmed, as indicated in FIG. 3D. Initially, the barb 330 is not deployed, such that the wire 300 is still easily retrievable for repositioning and redeployment, if necessary.

When the position of the J-shaped end 310 is confirmed to be in the desired location, the locking device 340 may be removed, as indicated in FIG. 3E. The deployment needle 140 may then be slidably pulled back, while substantially maintaining the wire 300 in the desired position, thereby deploying the barb 330, as indicated in FIG. 3F. With the barb 330 deployed, the needle 140, may then be completely removed, with the localization wire 300 securely retained in the desired position.

The localization wire 300 is made of a resilient material, with shape memory properties, such that the J-shaped end 310, and barb 330 may be constrained within the deployment needle 140, but will tend to their deployed shapes when the needle 140 is removed. A person skilled in the art will recognize that many materials with shape memory property would be useful in the present invention, including, but not limited to, stainless steel, nickel, titanium, and the alloy nitinol. In particular embodiments, the localization wire 300 is made of a shape memory metal, or of stainless steel.

The localization wire 300 has a diameter, which allows passage of the wire through a deployment needle 140 having an axial channel therethrough, and a hub 130 adapted to engage the locking device 340 of the localization wire 300. A current embodiment uses a 18 gauge deployment needle, although it will be obvious that other gauge needles may alternatively be used, and may be preferred in certain applications.

The localization wire 300 combines the advantages of the J-shaped Homer wire 100 and the barbed Kopans wire 200, discussed above. Moreover, the localization wire 300 overcomes these conventional wires' individual disadvantages. Because of the J-shaped end, the wire is retrievable or removable after the first stage of deployment if the position turns out to be suboptimal. The J-shaped end also prevents the wire form migration deeper into the tissue after it has been deployed in the desired location. The barb of the localization wire of the present invention allows the retention of the wire in tissue substantially without displacement during surgery. Also, the wire 300 includes a barb 330 that is disposed away from the distal end of the wire 300, which reduces the risk of the barb 330 breaking away from the wire 300 during use,

In another aspect, the present invention provides methods for deploying a preoperative localization wire.

In one embodiment, the method includes the steps of,

(a) providing a preoperative localization wire comprising:

a J-shaped end;

a branch point proximal to the J-shaped end, wherein a barb is attached to the wire at the branch point; and

a locking device distal to the J-shaped end;

(b) providing a localization needle having a hub at one end and a tip at the opposite end;

(c) loading the preoperative localization wire into the localization needle, wherein the preoperative localization wire is resilient, allowing passage of the localization wire through the localization needle, and wherein the J-shaped end and the barb of the localization wire are compressed;

(d) placing the localization needle into a tissue with the tip of the needle at a desired location;

(e) advancing the preoperative localization wire with the locking device until the locking device contacts the hub, wherein the hub accepts the locking device, and wherein the J-shaped end is deployed and the barb remains compressed;

(f) removing the locking device; and

(g) deploying the barb by pulling back the localization needle while fixing the localization wire.

The method of the present invention may further include the step of confirming the position of the localization wire to be at the desired location prior to removing the locking device. If necessary, the localization wire may be retracted and repositioned to ensure the wire at the desired location. After confirming the correct position of the localization wire, the locking device may be removed.

The localization wire 300 uses a four-step deployment process as shown in FIGS. 3C-3G. In the first step the deployment needle 140 is positioned at a desired location in the patient, with the J-shaped end 310 and the bard 330 of the wire 300 fully sheathed. In the second step, only the J-shaped end 310 is deployed while holding the deployment needle 140 steady, as shown in FIG. 3D. The J-shaped end 310 prevents the localization wire from advancing deeper into the tissue. However, at this step, the wire 300 is still retrievable for repositioning if the position of the wire in the tissue is not optimal. In the third step, after the suitability of the position of the J-shaped end 310 has been confirmed (FIG. 3E), the locking device 340 is removed and the barb 330 is deployed, as shown in FIG. 3F, by slidably retracting the deployment needle 140 along the wire 300, while the wire 300 remains in position. The deployed barb 330 secures the localization wire making inadvertent extraction of the wire 300 much more difficult. In the fourth step, the deployment needle 140 is fully extracted from the patient (FIG. 3G).

It is contemplated that the localization wire of the present invention can be used for various preoperative localization purposes. In one embodiment, the localization wire can be used for preoperative localization of breast lesions identified by X-ray, mammography, ultrasounds, CT, MRI, ultrasonography, or nuclear medicine. In one embodiment, the localization wire can be used for preoperative localization of small nodules in lung identified in a screening lung CT.

While illustrative embodiments have been illustrated and described, it will be appreciated that various changes can be made therein without departing from the spirit and scope of the invention.

The embodiments of the invention in which an exclusive property or privilege is claimed are defined as follows:

1. A preoperative localization wire, comprising:
a J-shaped end;
a branch point proximal to the J-shaped end, wherein a barb is attached to the wire at the branch point; and
a locking device distal to the J-shaped end;
wherein the wire is resilient, allowing passage of the wire through a deployment needle.

2. The preoperative localization wire of claim 1, wherein the J-shaped end has a radius from about 3 mm to about 6 mm.

3. The preoperative localization wire of claim 1, wherein the J-shaped end has a radius of about 5 mm.

4. The preoperative localization wire of claim 1, wherein the branch point is from about 2 mm to about 3 mm proximal to the J-shaped end bend.

5. The preoperative localization wire of claim 1, wherein wire is made of a material with shape memory property.

6. The preoperative localization wire of claim 1, wherein the wire is made of stainless steel.

7. The preoperative localization wire of claim 1, wherein the wire is made of shape memory alloy.

8. A method for deploying a preoperative localization wire, comprising:
(a) providing a localization wire comprising:
a J-shaped end;
a branch point proximal to the J-shaped end, wherein a barb is attached to the localization wire at the branch point; and
a locking device distal to the J-shaped end;
(b) providing a deployment needle having a bore therethrough, a hub at one end and a tip at the opposite end;
(c) slidably inserting the localization wire into the deployment needle bore such that the J-shaped end and the barb of the localization wire are sheathed by the deployment needle;
(d) inserting the deployment needle into a tissue to a desired location;
(e) advancing the localization wire until the locking device engages the hub, such that the J-shaped end is deployed and the barb remains sheathed;
(f) retracting the locking device; and
(g) deploying the barb by pulling back the deployment needle along the localization wire.

9. The method of claim 8, further comprising the step of removing the deployment needle from the tissue.

10. The method of claim 8, wherein the localization needle has a sharp tip.

11. The method of claim 8, wherein the deployment needle is an 18 gauge needle.

12. The method of claim 8, further comprising the step of confirming the position of the localization wire to be at the desired location prior to removing the locking device.

13. The method of claim 12, further comprising the step of retracting and repositioning the localization wire to ensure the localization wire at the desired location after confirming the position of the localization wire.
